(19) United States
(12) Patent Application Publication
(10) Pub. No.: US 2020/0337943 A1
(43) Pub. Date: Oct. 29, 2020

(54) CARDIOPULMONARY RESUSCITATION (CPR) SUPPORT BOARD

(71) Applicant: Mitchell J. Hargis, Kernersville, NC (US)

(72) Inventor: Mitchell J. Hargis, Kernersville, NC (US)

(21) Appl. No.: 16/396,891

(22) Filed: Apr. 29, 2019

(51) Int. Cl. A61H 31/00; A61G 1/04; A61N 1/39

(52) U.S. Cl. CPC A61H 31/005; A61G 1/04; A61N 1/39044; A61H 2201/1635; A61H 2203/0456; A61H 2201/5043; A61H 2201/10

(57) ABSTRACT

A support device for use when administering CPR includes a panel defining a pocket for receiving a defibrillator pad. A visible indicator is configured to provide visible indication to a CPR administrator. The visible indication includes a blinking light that blinks at a rate that approximates a desired CPR chest compression rate.

### TECHNICAL FIELD

The presently disclosed subject matter relates to a device for use with CPR.

### BACKGROUND

Cardiopulmonary resuscitation (CPR) is a well known and valuable method of first aid. CPR is used to resuscitate people who have suffered from cardiac arrest after heart attack, electric shock, chest injury and many other causes. During cardiac arrest, the heart stops pumping blood, and a person suffering cardiac arrest will soon suffer brain damage from lack of blood supply to the brain. Thus, CPR requires repetitive chest compression to squeeze the heart and the thoracic cavity to pump blood through the body. Very often, the patient is not breathing, and mouth to mouth artificial respiration or a bag valve mask is used to supply air to the lungs while the chest compression pumps blood through the body.

It has been widely noted that CPR and chest compression can save cardiac arrest patients, especially when applied immediately after cardiac arrest. Chest compression requires that the person providing chest compression repetitively push down on the sternum of the patient at 100 to 120 compressions per minute. CPR and closed chest compression can be used anywhere, wherever the cardiac arrest patient is stricken.

When a first aid provider performs chest compression effectively, blood flow in the body is typically about 25% to 30% of normal blood flow. This is enough blood flow to prevent brain damage. However, when chest compression is required for long periods of time, it is difficult if not impossible to maintain adequate compression of the heart and rib cage. Even experienced paramedics cannot maintain adequate chest compression for more than a few minutes. At the same time, it appears that, if chest compression could be adequately maintained, cardiac arrest victims could be sustained for extended periods of time. However, performing CPR on top of a gurney or stretcher is problematic because the gurney or stretcher, being formed of a padded material, gives when compressions are being applied, thus reducing the applied to the patient force. Additionally, applying compresses at the appropriate or desired rate is difficult if the rhythm is interrupted; it is even difficult training a CPR provider, particularly in instances where the user may slow down due to fatigue or where concentration is interrupted due to the hectic surroundings where visual and audio interruptions and distractions are prevalent. A device addressing these issues would be well-received in the medical community.

### SUMMARY

In some embodiments, the presently disclosed subject matter is directed to a support device for use when administering CPR. The support device includes a panel being positioned between a gurney or stretcher and a patient during administration of CPR to provide rigidity to the gurney or stretcher, and a visible indicator configured to provide visible indication to a CPR administrator. The visible indication includes a blinking light that blinks at a rate that approximates a desired CPR chest compression rate.

According to one or more embodiments, the panel defines a pocket for receiving a defibrillator pad.

According to one or more embodiments, the pocket is formed of a compliant material and is formed on an exterior facing surface of the panel.

According to one or more embodiments, the chest compression rate is about 100 to about 120 compressions per minute.

According to one or more embodiments, the pocket is defined on an exterior facing surface of the panel.

According to one or more embodiments, the panel defines a cutout for providing a gripping surface.

According to one or more embodiments, the visible indicator is defined on the panel. According to one or more embodiments, the visible indicator is defined on a same side of the panel as the pocket.

### BRIEF DESCRIPTION OF THE DRAWINGS

The previous summary and the following detailed descriptions are to be read in view of the drawings, which illustrate some (but not all) embodiments of the presently disclosed subject matter.

FIG. 1 is a front facing perspective view of a support device for use when administering CPR in accordance with some embodiments of the presently disclosed subject matter.

FIG. 2 is a perspective view of a defibrillator pad in accordance with some embodiments of the presently disclosed subject matter.

FIG. 3 is a perspective view of the support device stowed within a transport cart in accordance with some embodiments of the presently disclosed subject matter.

FIG. 4 is a top view of a support device for use when administering CPR in accordance with some embodiments of the presently disclosed subject matter.

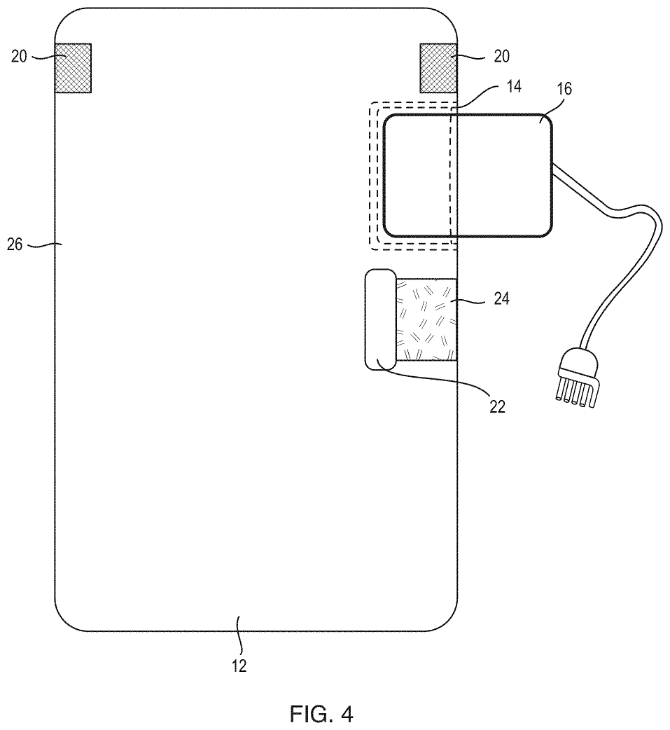

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

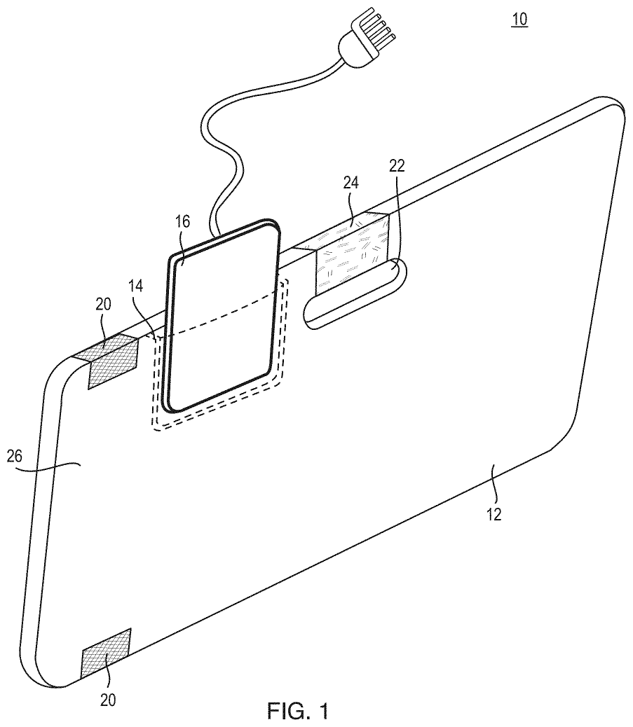

The presently disclosed subject matter is directed to a support device for use when administering CPR. The support device is generally designated 10 and is illustrated and shown in FIG. 1. Specifically, the support device 10 is provided for being positioned beneath a person on a gurney or stretcher to which CPR is being administered. Disadvantageously, in traditional CPR, the person to which CPR is being administered is on the soft padding of the gurney or stretcher and chest compressions cause upward and downward movement of the patient about the soft padding, reducing the applied transfer of forces to the patient. Since the amount of applied force is important for increasing blood flow rate, transferring force to the patient during chest compressions is vital to the success of the CPR treatment. As such, the device 10 may include a panel 12 that is placed under the patient and above the gurney or stretcher. In other words, the device 10 is placed between the patient and the gurney or stretcher to provide some rigidity to the gurney or stretcher during compressions.

The device 10 may include the panel 12 where the panel defines a pocket 14 for receiving a defibrillator pad 16. The pocket 14 may be formed of a compliant and flexible material and may extend from the panel 12; alternatively, the pocket 14 may be defined out of removal of material from panel 12. As illustrated, the pocket 14 may alternatively be defined on an exterior facing surface 26 of the panel 12.

The panel 12 may be made from a plastic material or any other material providing sufficient rigidity for purposes of providing support during chest compressions while administering CPR. The panel 12 may have any appropriately configured dimension and shape. For example, the panel 12 is shown with rounded edges and a certain thickness, but this depiction is for illustration purposes only.

The panel 12 advantageously includes a visible indicator or light 20 that is configured to provide visible indication to a CPR administrator. The visible indication includes a blinking light that blinks at a rate that approximates a desired CPR chest compression rate. In this manner, the blinking of the light tells the CPR administrator if they are applying compressions at the appropriate rate. Because the light 20 is positioned at a topward portion of the panel 12, the light is positioned within the line of site of a CPR administrator and will lead to positioning of the administrator's hands and head in a non-fatiguing posture. The light 20 is shown on each side of the panel 12, but may be positioned in any appropriate location. For example, the light 20 could be an array of lights that form around a perimeter of the panel 12. The light 20 may, however, be positioned in any appropriately configured place. Light 20 is considered advantageous over an audible alert or similar because the noise levels in an emergency situation are already elevated.

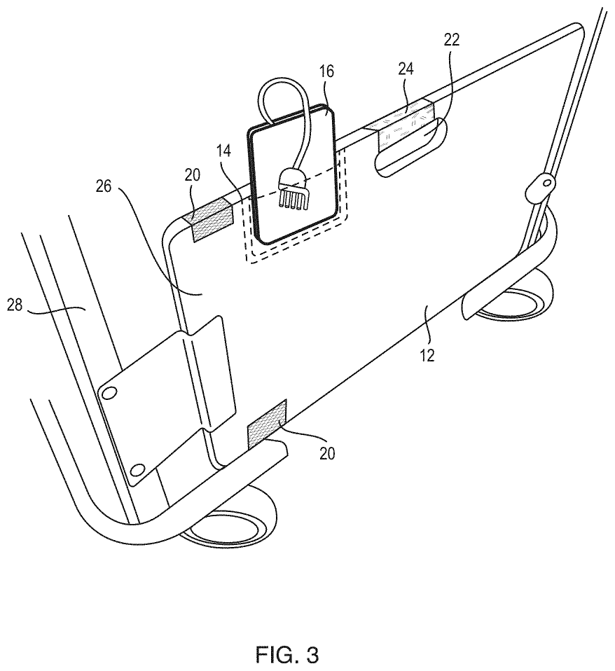

Light 20 may be configurable where the rate of blinking of the light is altered according to a desired input value provided by the operator. As an example, a normal desired rate of blinking may be 100 blinks per minute, corresponding to 100 compressions per minute. Alternatively, the light 20 may blink once for every five compressions or some other desired number. Light 20 may be integrated within the panel 12 as illustrated such that the light 20 doesn't extend beyond the periphery of the panel, or light 20 may extend from the periphery. The panel 12 may define a cutout 22 for providing a gripping surface 24. As illustrated in FIG. 3, the device 10 may be configured such that the surface 26 of the device is the same surface from which pocket 14 is defined.

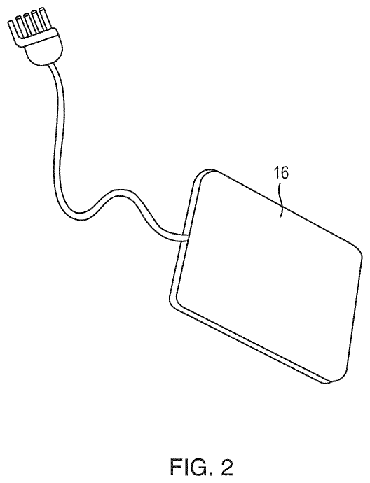

The defibrillator pad 16 is illustrated in FIG. 2. For the compliant type of pocket 14, different sizes of pads may be stowed. Alternatively, other tools or other items may be stowed within pocket 14. An area for wrapping cordage of the pad 6 may also be provided about the panel 12. The device 10 is configured for being stowed in a cart 28.

Thus a method of administering CPR is provided herein. The method includes positioning the support device 10 disclosed herein between a patient and a soft support surface. Alternatively, support device 10 may also be used between a patient and a rigid or firm support surface. If the defibrillator pads are needed, then those are removed and placed into use before placing the support device 10. The administrator then monitors the rate of visible indicators and adjusts their CPR administration rate in response to the rate of indicators.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A support device for use when administering CPR, the device comprising:
a panel being positioned between a gurney or stretcher and a patient during administration of CPR to provide rigidity to the gurney or stretcher;
a visible indicator configured to provide visible indication to a CPR administrator, the visible indication including a blinking light that blinks at a rate that approximates a desired CPR chest compression rate.

2. The device of claim 1, wherein the panel defines a pocket for receiving a defibrillator pad.

3. The device of claim 2, wherein the pocket is formed of a compliant material and is formed on an exterior facing surface of the panel.

4. The device of claim 1, wherein the chest compression rate is about 100 to about 120 compressions per minute.

5. The device of claim 2, wherein the pocket is defined on an exterior facing surface of the panel.

6. The device of claim 1, wherein the panel defines a cutout for providing a gripping surface.

7. The device of claim 1, wherein the visible indicator is defined on the panel.

8. The device of claim 2, wherein the visible indicator is defined on a same side of the panel as the pocket.

9. A support device for use when administering CPR, the device comprising:
a panel formed of a rigid material and being positioned between a gurney or stretcher and a patient during administration of CPR to provide rigidity to the gurney or stretcher;
a visible indicator configured to provide visible indication to a CPR administrator, the visible indication including a blinking light that blinks at a rate that approximates a desired CPR chest compression rate,
wherein the panel defines a pocket for receiving a defibrillator pad,
wherein the pocket is formed of a compliant material and is formed on an exterior facing surface of the panel.

10. The device of claim 9, wherein the chest compression rate is about 100 to about 120 compressions per minute.

11. The device of claim 9, wherein the pocket is defined on an exterior facing surface of the panel.

12. The device of claim 9, wherein the panel defines a cutout for providing a gripping surface.

13. The device of claim 9, wherein the visible indicator is defined on the panel.

14. The device of claim 9, wherein the visible indicator is defined on a same side of the panel as the pocket.
